(19) United States
(12) Patent Application Publication
(10) Pub. No.: US 2020/0196866 A1
(43) Pub. Date: Jun. 25, 2020

(54) NON-CONTACT VITAL-SIGN MONITORING SYSTEM AND METHOD

(71) Applicant: Wistron Corporation, New Taipei City (TW)

(72) Inventors: Sheng-Lun Chiou, New Taipei City (TW); Yin-Yu Chen, New Taipei City (TW); Fang-Ming Wu, New Taipei City (TW); Ching-Han Chang, New Taipei City (TW)

(21) Appl. No.: 16/280,774

(22) Filed: Feb. 20, 2019

(30) Foreign Application Priority Data
Dec. 21, 2018 (TW) 107146296

(51) Int. Cl. A61B 5/00; A61B 5/08; A61B 5/05; A61B 5/02; G01S 7/00

(52) U.S. Cl. CPC A61B 5/0022; A61B 5/0816; A61B 5/0507; A61B 5/02; G01S 13/88; G01S 7/003; A61B 5/7257; A61B 5/6823; A61B 5/7225

(57) ABSTRACT

A non-contact vital-sign monitoring system having a radar disposed in vicinity of a monitored subject includes a data buffer storing output signals of the radar sampled in sequence during a predetermined period; a status classifier determining status of the monitored subject; and a vital-sign detector determining a vital sign of the monitored subject according to the output signals of stationary status.

### CROSS-REFERENCE TO RELATED APPLICATIONS

This application claims priority of Taiwan Patent Application No. 107146296, filed on Dec. 21, 2018, the entire contents of which are hereby incorporated by reference.

### BACKGROUND OF THE INVENTION

### 1. Field of the Invention

The present invention generally relates to a monitoring system, and more particularly to a non-contact vital-sign monitoring system and method.

### 2. Description of Related Art

Body temperature (BT), blood pressure (BP), heart rate (HR) and respiratory rate (RR) are four primary vital signs. The detection and measurement of the vital signs may be used to evaluate health condition or a clue to illness of a person.

Most conventional health monitoring devices are contact type, and contact with the body of a monitored subject via wires. Accordingly, the contact health monitoring devices would restrict the movement of the monitored subject. Moreover, the contact health monitoring devices may only be operated by trained personnel.

A professional is generally required to make observation and recording while using conventional (contact or non-contact) health monitoring devices. Due to limited manpower, health measure can only be made at intervals. Therefore, a detecting chance may probably be lost in case of emergency, and an opportunity to save life may be missed. A need has thus arisen to propose an all-day non-contact vital-sign monitoring system.

### SUMMARY OF THE INVENTION

In view of the foregoing, it is an object of the embodiment of the present invention to provide a non-contact vital-sign monitoring system and method for detecting a respiratory rate and a heart rate.

According to one embodiment, a non-contact vital-sign monitoring system having a radar disposed in vicinity of a monitored subject includes a data buffer, a status classifier and a vital-sign detector. The data buffer stores output signals of the radar sampled in sequence during a predetermined period. The status classifier determines status of the monitored subject according to the output signals. The vital-sign detector determines a vital sign of the monitored subject according to the output signals of stationary status.

### BRIEF DESCRIPTION OF THE DRAWINGS

FIG. 1 shows a block diagram illustrating a non-contact vital-sign monitoring system according to one embodiment of the present invention;

FIG. 2 shows a flow diagram illustrating a non-contact vital-sign monitoring method for determining vital-sign status according to one embodiment of the present invention;

FIG. 3A shows a flow diagram illustrating a method of detecting a respiratory rate according to one embodiment of the present invention;

FIG. 3B shows a flow diagram illustrating a method of detecting a heart rate according to one embodiment of the present invention;

FIG. 4 shows a flow diagram illustrating a non-contact vital-sign monitoring method according to one embodiment of the present invention; and

FIG. 5 shows a block diagram illustrating a non-contact vital-sign monitoring system according to another embodiment of the present invention.

### DETAILED DESCRIPTION OF THE INVENTION

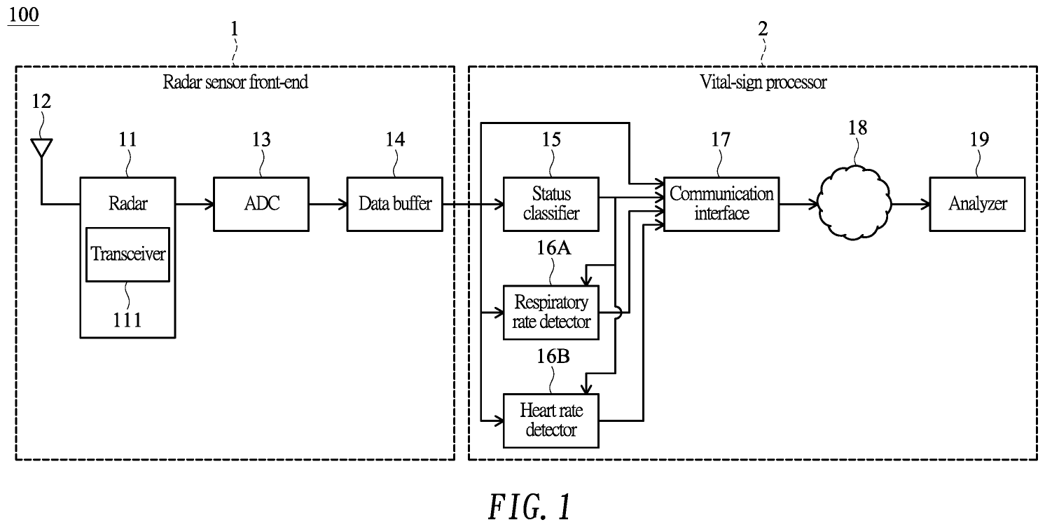

FIG. 1 shows a block diagram illustrating a non-contact vital-sign monitoring system 100 adaptable to monitoring a vital sign, such as a heart rate or a respiratory rate, according to one embodiment of the present invention.

In the embodiment, the non-contact vital-sign monitoring system (monitoring system hereinafter) 100 may include a radar sensor front-end 1 that may include a radar 11, such as a continuous-wave (CW) radar, disposed in vicinity of a monitored subject covered by the detection range of the radar 11. In one embodiment, the radar 11 may be disposed above the chest of the monitored subject lying on a bed. In another embodiment, the radar 11 may be disposed in other orientation such as below or beside the bed. In one embodiment, the radar 11 may include an ultra-wideband (UWB) radar such as frequency modulated continuous waveform (FMCW) radar. The radar sensor front-end 1 of the monitoring system 100 may include an antenna 12 electrically coupled to the radar 11, and configured to transmit radio-frequency (RF) signals and to receive reflected RF signals. The radar 11 of the embodiment may include a transceiver 111 configured to generate baseband output signals according to the reflected RF signals. In another embodiment, the antenna 12 may be integrated with the radar 11. In a further embodiment, the radar sensor front-end 1 may include multiple radars 11 disposed at different locations.

The radar sensor front-end 1 of the monitoring system 100 of the embodiment may include an analog-to-digital converter (ADC) 13 coupled to receive the (analog) output signals from the radar 11, and configured to convert the analog output signals into digital output signals. In the embodiment, the output signal of the radar 11 may include an in-phase polarization signal (in-phase signal hereinafter) I and a quadrature polarization signal (quadrature signal hereinafter) Q.

The radar sensor front-end 1 of the monitoring system 100 may include a data buffer 14 configured to store the output signals sampled in sequence during a predetermined period. For example, the data buffer 14 may store eight pieces of output signals sampled at intervals each of 2.5 seconds. Accordingly, the data buffer 14 may store the output signals spanning a period of 20 seconds. The spanning period, for example, of 10, 30 or 60 seconds may be set according to specific applications. The sampling interval may be determined based on response time. The shorter the sampling interval is, the more rapidly the monitoring system 100 responds.

In the embodiment, the monitoring system 100 may include a vital-sign processor 2 that may include a status classifier 15 configured to determine status of the monitored subject according to the output signals of the radar 11. In the embodiment, status of the monitored subject may, but not necessarily, be classified into three types: stationary, motion and no vital-sign. Stationary status may indicate that the monitored subject sleeps or rests, motion status may indicate that the monitored subject turns or moves, and no vital-sign status may indicate that the monitored subject is not currently on the bed. Specifically, for example, stationary status may indicate that the monitored subject turns to a posture during sleep, stays still while watching television, or slightly trembles; motion status may indicate that the monitored subject moves on bed or off bed, or moves around the bed; and no vital-sign status may indicate that the monitored subject is no longer vital. In another embodiment, the status classifier 15 may receive output signals of multiple radars 11 disposed at different locations.

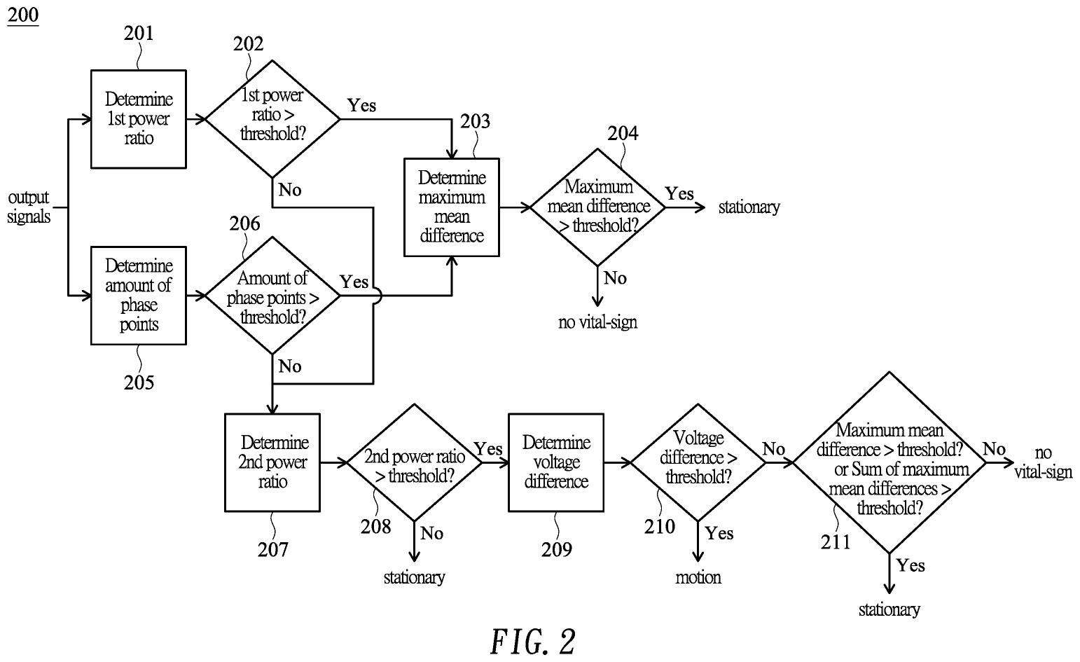

FIG. 2 shows a flow diagram illustrating a non-contact vital-sign monitoring method (method hereinafter) 200 for determining vital-sign status and adaptable to the status classifier 15 of FIG. 1 according to one embodiment of the present invention. In step 201, a first power ratio in frequency domain is determined according to the output signals of the radar 11, for example, by using fast Fourier transform (FFT) algorithm. In the embodiment, the first power ratio is defined as a ratio of power in a predetermined (first) frequency range (e.g., 12.5-25 Hz) to total power, and may be expressed as:

first power ratio=P_{fr1}/Pₜ

where P_{fr1} represents power in the predetermined (first) frequency range, and Pₜ represents total power (in a full frequency range).

Next, in step 202, it is determined whether the first power ratio is greater than a predetermined (first) threshold.

If determination in step 202 is positive, the flow goes to step 203 to determine a maximum mean difference, which represents a maximum among a plurality of mean differences. In the embodiment, the data buffer 14 may have a total mean M. The data buffer 14 may be divided into a plurality of (e.g., m) blocks, each having a divisional mean DM₁-DMₘ. The mean difference is defined as (the absolute value of) a difference of the divisional mean and the total mean, that is, DMₓ−M, x is 1 to m. The maximum mean difference may be expressed as:

maximum mean difference=max{abs[(DM₁,DM₂, . . . DMₘ)−(M,M, . . . M)}

where abs( ) represents absolute value function, and max( ) represents maximum value function.

In step 204, it is determined whether the maximum mean difference is greater than a predetermined (second) threshold. If determination in step 204 is positive, the monitored subject is determined or predicted as being in stationary status; otherwise the monitored subject is determined or predicted as being in no vital-sign status.

On the other hand, the method 200 for determining vital-sign status of the embodiment may include steps 205-206 to be executed concurrently with step 201-202. In step 205, an amount of phase point is determined according to the output signals of the radar 11. In the embodiment, the in-phase signal I, the quadrature signal Q and phase φ may have the following relationship:

I(n)=A_{I}(n)cos(p(n)+θ)

Q(n)=A_{Q}(n)sin(p(n)+θ)

φ=arctan[Q(n)/I(n)]

In the embodiment, if the phase φ is within a predetermined range (e.g., between 44.5° and 45.51°), it is then numbered among the phase points. Next, in step 206, it is determined whether the amount of phase points is greater than a predetermined (third) threshold.

If determination in step 206 is positive, the flow goes to step 203 to determine a maximum mean difference. In step 204, it is determined whether the maximum mean difference is greater than a predetermined (second) threshold. If determination in step 204 is positive, the monitored subject is determined or predicted as being in stationary status; otherwise the monitored subject is determined or predicted as being in no vital-sign status.

If determination in step 202 or step 206 is negative, the flow goes to step 207 to determine a second power ratio in frequency domain according to the output signals of the radar 11, for example, by using fast Fourier transform (FFT) algorithm. In the embodiment, the second power ratio is defined as a ratio of power in a predetermined (second) frequency range (e.g., 3-25 Hz) to total power, and may be expressed as:

second power ratio=P_{fr2}/Pₜ

where P_{fr2} represents power in the predetermined (second) frequency range, and Pₜ represents total power (in a full frequency range). It is noted that the predetermined second frequency range may be equal to, or different from the predetermined first frequency range.

Next, in step 208, it is determined whether the second power ratio is greater than a predetermined (fourth) threshold, which may be equal to, or different from the predetermined (first) threshold. If determination in step 208 is negative, the monitored subject is determined or predicted as being in stationary status; otherwise the flow goes to step 209. If the predetermined second frequency range is equal to the predetermined first frequency range, and the predetermined fourth threshold is equal to the predetermined first threshold, steps 207-208 may be omitted for the reason that steps 207-208 are duplicates of steps 201-202.

In step 209, a voltage difference, such as point-to-point or peak-to-peak voltage difference, of the output signal is determined. Next, in step 210, it is determined whether the voltage difference is greater than a predetermined (fifth) threshold. If determination in step 210 is positive, the monitored subject is determined or predicted as being in motion status; otherwise the flow goes to step 211.

In step 211, it is determined whether the maximum mean difference is greater than a predetermined (second) threshold (similar to step 204), or whether a sum of maximum mean differences is greater than a predetermined (sixth) threshold. If at least one of determinations is positive, the monitored subject is determined or predicted as being in stationary status; otherwise the monitored subject is determined or predicted as being in no vital-sign status. In the embodiment, the sum of maximum mean differences is a sum of maximum mean difference of the in-phase signal I and maximum mean difference of the quadrature signal Q.

Referring back to FIG. 1, the vital-sign processor 2 of the monitoring system 100 of the embodiment may include a respiratory rate detector 16A coupled to receive the status determined by the status classifier 15 and configured to obtain a respiratory rate of the monitored subject according to the output signals (including the in-phase signal I and the quadrature signals Q) of the radar 11 of stationary status. As stated above, the data buffer 14 may store eight pieces of output signals sampled at intervals each of 2.5 seconds in the embodiment. While determining the respiratory rate, if the monitored subject is determined or predicted as being not in stationary status, corresponding data in the data buffer 14 may be set a predetermined value, such as a direct-current (DC) voltage value of the output signal. In another embodiment, the predetermined value may be a fixed waveform composed of one or more frequencies. In a further embodiment, the predetermined value may be a linear-nonlinear computation result according to preceding data stored in the data buffer 14.

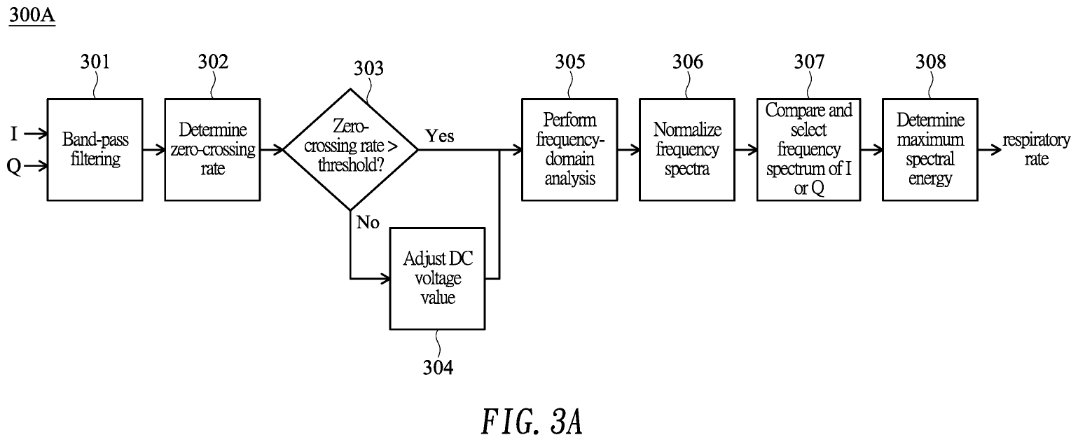

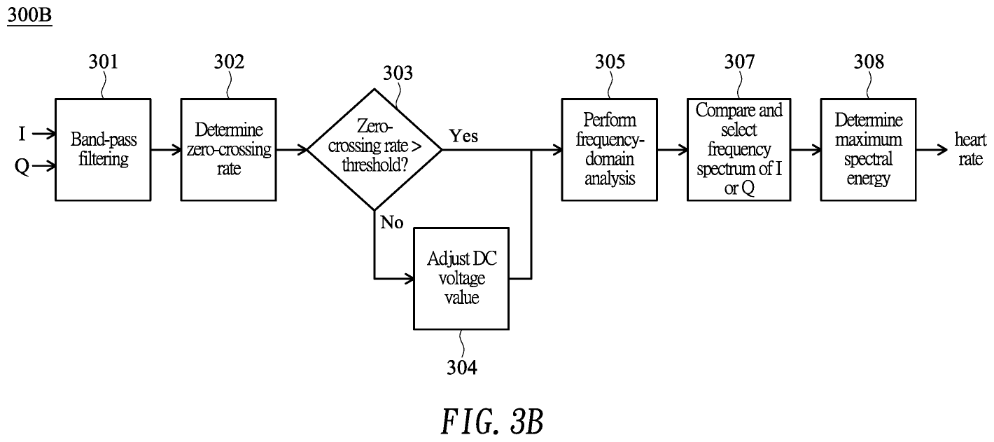

FIG. 3A shows a flow diagram illustrating a method 300A of detecting a respiratory rate adaptable to the respiratory rate detector 16A of FIG. 1 according to one embodiment of the present invention. In step 301, the output signals (including the in-phase signals I and the quadrature signals Q) of the radar 11 are subjected to band-pass filtering by a band-pass filter, thereby generating filtered signals. In one embodiment, a passband frequency range of the band-pass filter corresponds to a frequency range of respiratory rate, for example but not limited to, 0.16-0.8 Hz.

In step 302, a zero-crossing rate in time domain is determined according to the filtered signals from the band-pass filter. In the embodiment, zero-crossing refers to an intersection between an alternative-current (AC) component of the output signal and a direct-current (DC) component of the output signal. When the output signal is normal, the zero-crossing rate is large. However, when the output signal is abnormal (e.g., the AC component shifts up or down), the zero-crossing rate becomes small.

Next, in step 303, it is determined whether the zero-crossing rate is greater than a predetermined (seventh) threshold. If determination in step 303 is negative (indicating that the AC component of the output signal shifts), the flow goes to step 304 to adjust a DC voltage value of the output signal.

If determination in step 303 is positive or step 304 finishes, the flow goes to step 305 to perform frequency-domain analysis, according to which a frequency spectrum (of energy distribution) of the in-phase signal I and a frequency spectrum (of energy distribution) of the quadrature signal Q are obtained. Next, in step 306, the frequency spectra of the in-phase signal I and the quadrature signal Q are normalized.

In step 307, a maximum spectral energy of the in-phase signal I and a maximum spectral energy of the quadrature signal Q are compared. Accordingly, the frequency spectrum of the in-phase signal I or the frequency spectrum of the quadrature signal Q is selected. In other words, the frequency spectrum of the in-phase signal I is selected if the maximum spectral energy of the in-phase signal I is greater than the maximum spectral energy of the quadrature signal Q; otherwise the frequency spectrum of the quadrature signal Q is selected. In step 308, a maximum spectral energy of the selected frequency spectrum is determined, and a corresponding frequency is determined as the respiratory rate.

Referring back to FIG. 1, the monitoring system 100 of the embodiment may include a heart rate detector 16B coupled to receive the status determined by the status classifier 15 and configured to obtain a heart rate of the monitored subject according to the output signals (including the in-phase signal I and the quadrature signals Q) of the radar 11 of stationary status.

FIG. 3B shows a flow diagram illustrating a method 300B of detecting a heart rate adaptable to the heart rate detector 16B of FIG. 1 according to one embodiment of the present invention. The flow of FIG. 3B is similar to the flow of FIG. 3A, and corresponding steps are thus designated with the same numerals, difference of which will be described below.

In step 301, a passband frequency of the method 300B for detecting the heart rate is higher than (or equal to) the passband frequency of the method 300A for detecting the respiratory rate. In one embodiment, a passband frequency range of the method 300B for detecting the heart rate corresponds to a frequency range of heart rate, for example but not limited to, 0.7-3 Hz. In the method 300B for detecting the heart rate, normalization on the frequency spectra in step 306 may be omitted. In step 308, a maximum spectral energy of the selected frequency spectrum is determined, and a corresponding frequency is determined as the heart rate.

Referring back to FIG. 1, the vital-sign processor 2 of the monitoring system 100 of the embodiment may include a communication interface 17 configured to transfer the output signals of the radar 11, the status determined by the status classifier 15, the respiratory rate of the monitored subject detected by the respiratory rate detector 16A and/or the heart rate of the monitored subject detected by the heart rate detector 16B to an analyzer 19 via a network 18 (e.g., the Internet). The communication interface 17 may be a wired communication interface such as universal asynchronous receiver-transmitter (UART), Inter-Integrated Circuit (I2C), Serial Peripheral Interface (SPI), Controller Area Network (CAN), Recommended Standard (RS) 232 or Recommended Standard (RS) 422; may be a wireless communication interface such as Wireless Sensor Network (e.g., EnOcean, Bluetooth or ZigBee); may be a cellular network such as second generation (2G), third generation (3G), Long-Term Evolution (LTE), or fifth generation (5G); may be a wireless local network such as WLAN or Worldwide Interoperability for Microwave Access (WiMAX); or may be a short range point-to-point communication such as Radio-frequency identification (RFID), EnOcean or Near-field communication. In another embodiment, the communication interface 17 is configured to transfer the output signals of the radar 11, the status determined by the status classifier 15, the respiratory rate of the monitored subject detected by the respiratory rate detector 16A and/or the heart rate of the monitored subject detected by the heart rate detector 16B to the analyzer 19 via a signal cable.

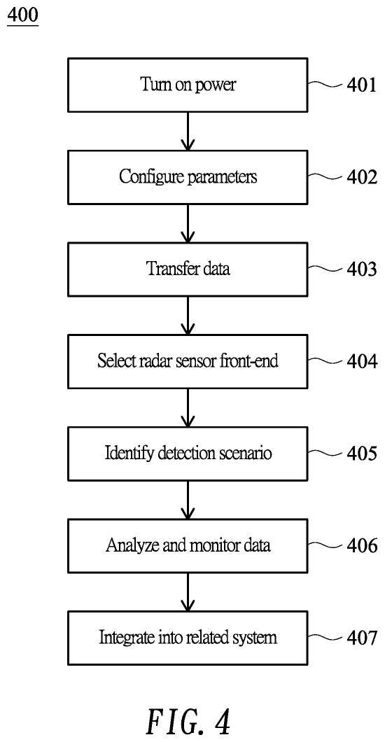

FIG. 4 shows a flow diagram illustrating a non-contact vital-sign monitoring method 400 according to one embodiment of the present invention. In step 401, power of the radar 11, the ADC 13, the data buffer 14, the status classifier 15, the respiratory rate detector 16A and the heart rate detector 16B is turned on. In the embodiment, the radar 11, the ADC 13, the data buffer 14, the status classifier 15, the respiratory rate detector 16A and the heart rate detector 16B may be implemented, for example, by a digital signal processor (DSP).

In step 402, primary parameters (e.g., division of the data buffer 14, the (first) frequency range (step 201), the (second) frequency range (step 207) and the passband frequency range (step 301)) of the data buffer 14, the status classifier 15, the respiratory rate detector 16A and the heart rate detector 16B are configured. In step 403, the communication interface 17 transfers the output signals, the status, the respiratory rate and/or the heart rate to the analyzer 19 (which may be disposed in cloud) via the network 18.

Next, in step 404, the analyzer 19 may select one among plural radar sensor front-ends 1. In step 405, a detection scenario (e.g., the monitored subject lies down on one's back, reclines, or lies down on one's side) is identified. In step 406, the transferred data of the radar sensor front-end 1 are analyzed and monitored. A warning may be issued to predetermined personnel or units when an emergent situation happens. In step 407, analysis result may be integrated into a related system (e.g., hospital system) for obtaining general and adequate judgement and comprehension.

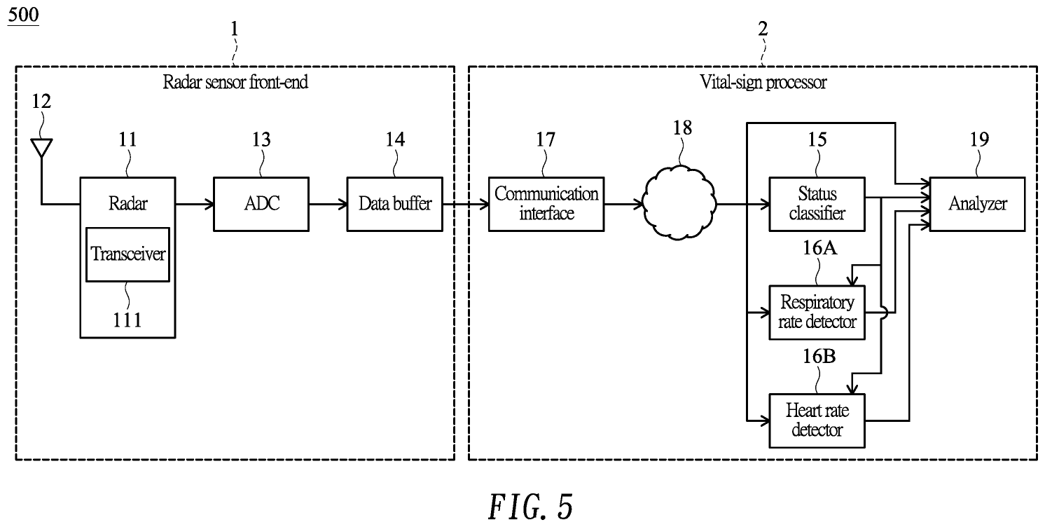

FIG. 5 shows a block diagram illustrating a non-contact vital-sign monitoring system (monitoring system hereinafter) 500 according to another embodiment of the present invention. The monitoring system 500 of FIG. 5 is similar to the monitoring system 100 of FIG. 1 with the following exception. In the embodiment, the communication interface 17 of the monitoring system 500 may receive the output signals of the radar 11 via the data buffer 14, and the output signals of the radar 11 may then be transferred to the status classifier 15, the respiratory rate detector 16A and/or the heart rate detector 16B via the network 18 (or directly). Accordingly, in the present embodiment, only the radar sensor front-end 1 (i.e., the radar 11, the antenna 12, the ADC 13 and the data buffer 14), but not the status classifier 15, the respiratory rate detector 16A and the heart rate detector 16B, need be installed in each ward.

Although specific embodiments have been illustrated and described, it will be appreciated by those skilled in the art that various modifications may be made without departing from the scope of the present invention, which is intended to be limited solely by the appended claims.

What is claimed is:

1. A non-contact vital-sign monitoring system having a radar disposed in vicinity of a monitored subject, the system comprising:
a data buffer storing output signals of the radar sampled in sequence during a predetermined period;
a status classifier determining status of the monitored subject according to the output signals; and
a vital-sign detector determining a vital sign of the monitored subject according to the output signals of stationary status.

2. The system of claim 1, wherein the radar comprises a continuous-wave radar or an ultra-wideband radar.

3. The system of claim 1, wherein corresponding data in the data buffer is set a predetermined value when the monitored subject is not in stationary status.

4. The system of claim 1, wherein the status classifier determines status of the monitored subject according to one or more power ratios, an amount of phase points or one or more voltage differences of the output signals.

5. The system of claim 1, wherein the vital-sign detector comprises a respiratory rate detector.

6. The system of claim 5, wherein the respiratory rate detector receives the status determined by the status classifier and determines a respiratory rate of the monitored subject according to a frequency associated with a maximum spectral energy of the output signals of stationary status.

7. The system of claim 1, wherein the vital-sign detector comprises a heart rate detector.

8. The system of claim 7, wherein the heart rate detector receives the status determined by the status classifier and determines a heart rate of the monitored subject according to a frequency associated with a maximum spectral energy of the output signals of stationary status.

9. The system of claim 1, further comprising:
an analyzer; and
a communication interface that transfers the output signals, the status or the vital sign to the analyzer.

10. A non-contact vital-sign monitoring method, comprising:
determining a first power ratio in frequency domain according to a plurality of buffered output signals of a radar that is disposed in vicinity of a monitored subject, and determining whether the first power ratio is greater than a predetermined first threshold;
determining an amount of phase points according to the output signals, and determining whether the amount of phase points is greater than a predetermined third threshold;
determining a maximum mean difference according to the output signals if the first power ratio is greater than the first threshold and the amount of phase points is greater than the third threshold, and determining whether the maximum mean difference is greater than a predetermined second threshold;
determining a second power ratio in frequency domain according to the output signals if the first power ratio is not greater than the first threshold or the amount of phase points is not greater than the third threshold, and determining whether the second power ratio is greater than a predetermined fourth threshold; and
determining a voltage difference according to the output signals, and determining whether the voltage difference is greater than a predetermined fifth threshold.

11. The method of claim 10, wherein the monitored subject is determined as being in stationary status if the maximum mean difference is greater than the second threshold, otherwise the monitored subject is determined as being in no vital-sign status.

12. The method of claim 10, wherein the monitored subject is determined as being in stationary status if the second power ratio is not greater than the fourth threshold.

13. The method of claim 10, wherein the monitored subject is determined as being in motion status if the voltage difference is greater than the fifth threshold.

14. The method of claim 10, further comprising;
determining a sum of a plurality of the maximum mean differences according to the output signals, and determining whether the sum of the maximum mean differences is greater than a predetermined sixth threshold.

15. The method of claim 14, wherein the monitored subject is determined as being in stationary status if the voltage difference is not greater than the fifth threshold and the sum of the maximum mean differences is greater than the sixth threshold, otherwise the monitored subject is determined as being in no vital-sign status.

16. A non-contact vital-sign monitoring method, comprising:
band-pass filtering output signals of a radar to generate filtered signals including in-phase signals and quadrature signals;
determining a zero-crossing rate in time domain according to the filtered signals, and determining whether the zero-crossing rate is greater than a predetermined threshold;
obtaining a frequency spectrum of the in-phase signals and a frequency spectrum of the quadrature signals according to the filtered signals;
selecting the frequency spectrum of the in-phase signals or the frequency spectrum of the quadrature signals as a selected frequency spectrum by comparing a maximum spectral energy of the in-phase signals and a maximum spectral energy of the quadrature signals; and
determining a maximum spectral energy of the selected frequency spectrum, a frequency associated with the maximum spectral energy of the selected frequency spectrum being determined as a vital sign.

17. The method of claim 16, further comprising:
adjusting a direct-current voltage value of the output signals if the zero-crossing rate is not greater than the threshold.

18. The method of claim 16, further comprising:
normalizing the frequency spectrum of the in-phase signals and the frequency spectrum of the quadrature signals.

19. The method of claim 16, wherein the band-pass filtering has a passband frequency range corresponding to a frequency range of respiratory rate, and the vital sign represents a respiratory rate of a monitored subject; or the band-pass filtering has a passband frequency range corresponding to a frequency range of heart rate, and the vital sign represents a heart rate of the monitored subject.
